(19) United States
(12) Patent Application Publication
(10) Pub. No.: US 2026/0263663 A1
(43) Pub. Date: Sep. 10, 2026

(54) MULTI-LAYER ELECTROSPUN MICRO/NANOFIBROUS MEMBRANES BASED ON MARINE SULFATED POLYSACCHARIDES FOR PERIODONTAL TISSUE/BONE REGENERATION

(71) Applicants: Uni-Pharma Kleon Tsetis Pharmaceutical Laboratories S.A., Kifissia Attikis (GR); National and Kapodistrian University of Athens, Athens (GR)

(72) Inventors: Vasileios ROUSSIS, Athens (GR); Efstathia IOANNOU, Athens (GR); Stefanos KIKIONIS, Athens (GR); Konstantina ILIOU, Athens (GR); Ermolaos IATROU, Athens (GR); Iannis CHOINOPOULOS, Athens (GR); Spyridon ZINELIS, Athens (GR); Georgios ELIADES, Athens (GR); Efthymia KITRAKI, Athens (GR); Georgios POLYCHRONIS, Athens (GR); Aikaterini KARRA, Athens (GR); Ioulia TSETI, Kifissa Attikis (GR)

(73) Assignees: Uni-Pharma Kleon Tsetis Pharmaceutical Laboratories S.A., Kifissia Attikis (GR); National and Kapodistrian University of Athens, Athens (GR)

(21) Appl. No.: 19/165,901

(22) PCT Filed: Mar. 15, 2024
(86) PCT No.: PCT/EP2024/056923

§ 371 Date: Sep. 16, 2025

(30) Foreign Application Priority Data
Mar. 17, 2023 (GR) 20230100221

(51) Int. Cl. A61L 31/04; A61C 8/02; A61L 31/14; A61L 31/16; D04H 1/728

(52) U.S. Cl. CPC A61L 31/041; A61C 8/0006; A61L 31/042; A61L 31/146; A61L 31/148; A61L 31/16; D04H 1/728; A61L 2300/406; A61L 2400/12; A61L 2430/02; A61L 2430/34

(57) ABSTRACT

The present invention refers to compositions in the form of a two- or three-layer membrane for periodontal use, which comprise an outer layer of hydrophobic polymers and one or two inner layers in the form of electrospun micro/nanofibers based on marine sulfated polysaccharides and calcium.

### DESCRIPTION OF THE INVENTION

The present invention refers to compositions in the form of a two- or three-layer membrane for periodontal use, which comprise an outer layer of hydrophobic polymers and one or two inner layers in the form of electrospun micro/nanofibers based on marine sulfated polysaccharides and calcium.

Periodontitis is a microbially-induced inflammation of the periodontium [D. Fraser, et al. Periodontal Wound Healing and Regeneration: Insights for Engineering New Therapeutic Approaches. Front. Dent. Med. 2022, 3, 815810], characterized by the destruction of periodontal ligament (PDL) and alveolar bone [V. Uskokovid et al. Polymeric nanotechnologies for the treatment of periodontitis: A chronological review. Int. J. Pharm. 2022, 625, 122065] and it constitutes the principal cause of teeth loss in adults.

The optimal target of reestablishing tooth functionality is achieved by periodontal regeneration procedures that aim to restore the periodontal apparatus to its initial state [Uskoković V. et al. Polymeric nanotechnologies for the treatment of periodontitis: A chronological review. Int. J. Pharm. 2022, 625, 122065; S. Mirzaeei, et al. An overview of guided tissue regeneration (GTR) systems designed and developed as drug carriers for management of periodontitis. J. Drug Deliv. Sci. Technol. 2022, 71, 103341]. Guided tissue/bone regeneration (GTR/GBR) membranes act as a physical barrier isolating the fast-growing epithelium and providing adequate time and space for the PDL cells and osteoblasts to proliferate and counter affect attachment loss [D. Fraser, et al. Periodontal Wound Healing and Regeneration: Insights for Engineering New Therapeutic Approaches. Front. Dent. Med. 2022, 3, 815810; S. Mirzaeei, et al. An overview of guided tissue regeneration (GTR) systems designed and developed as drug carriers for management of periodontitis. J. Drug Deliv. Sci. Technol. 2022, 71, 103341].

However, the major drawback of the currently available GTR/GBR membranes is their low attachment rates and the unsuccessful regeneration of bone cells.

Nanotechnology can contribute to periodontal tissue engineering [Uskoković V. et al. Polymeric nanotechnologies for the treatment of periodontitis: A chronological review. Int. J. Pharm. 2022, 625, 122065; S. Mirzaeei, et al. An overview of guided tissue regeneration (GTR) systems designed and developed as drug carriers for management of periodontitis. J. Drug Deliv. Sci. Technol. 2022, 71, 103341], as it has been proven that the nanofibrous scaffolds/membranes that are designed to resemble the natural architecture of the extracellular matrix (ECM) enhance cell proliferation and differentiation and are nowadays developed as carriers for cells, growth factors, antiseptic/antimicrobial and other bioactive substances [S. Zupandid, et al. Sustained release of antimicrobials from double-layer nanofiber mats for local treatment of periodontal disease, evaluated using a new micro flow through apparatus. J. Control. Release 2019, 316, 223-235]. Electrospinning, using an electrically charged jet of polymer solution, is the most successful method used for the preparation of fibrous scaffolds, with fiber diameters ranging in the micro- and nano-scale (sizes of the order of μm to nm), and shows great prospects in the development of membranes for periodontal regeneration [S-L. Bee, et al. Asymmetric resorbable-based dental barrier membrane for periodontal guided tissue regeneration and guided bone regeneration: A review. J. Biomed. Mater. Res. 2022, 110, 2157-2182]. It is an efficient, versatile, simple, cost-effective and up-scalable method for the production of polymeric fibers, offering the possibility to produce fibers with tailor-made properties and diverse morphologies by changing a wide range of parameters (e.g., applied voltage, polymer flow rate, tip-to-collector distance).

The major advantages of micro/nanofibrous membranes are that they possess high porosity, increased surface area, tunable size, fiber orientation and diameter, as well as tunable micromechanical properties [M. Zafar, et al. Potential of Electrospun Nanofibers for Biomedical and Dental Applications. Materials 2016, 9, 1-21]. Furthermore, their intrinsic interconnectivity and topography can facilitate the vascularization and transport of nutrients and bioactive agents [J. Park, et al. Nanosize and vitality: TiO2 nanotube diameter directs cell fate. Nano Lett. 2007, 7, 1686-1691]. In particular, the nanotopography of their fibrous structure increases the total surface area, surface-to-volume ratio and roughness that in turn enhance the adhesion between the bony-cemental interface and the underlying scaffold surfaces, whereas the microtopography facilitates cell penetration, vascularization, and diffusion of nutrients and offers better spatial organization for cell growth [X. Xu, et al. Biodegradable engineered fiber scaffolds fabricated by electrospinning for periodontal tissue regeneration. J. Biomater. Appl. 2021, 36, 55-75].

However, the main drawback of the currently available micro/nanofibrous membranes is that they do not promote the regeneration of bone cells and periodontal ligament cells.

Document US2018/0078346A1 refers to compositions of three-layer dental membranes from electrospun nanofibers of biodegradable polymers, bearing pores of different diameters. However, the formulation of document US2018/0078346A1 is based on synthetic polymers and hydrophilic agents that do not have the ability to promote the regeneration of bone cells and periodontal ligament cells. Also, the formulation of document US2018/0078346A1 does not include marine sulfated polysaccharides nor calcium.

Marine sulfated polysaccharides are highly biocompatible and biodegradable materials featuring diverse structures and functionalities [M. J. Cardoso, et al. Marine origin polysaccharides in drug delivery systems. Mar. Drugs 2016, 14, 34]. The wide spectrum of bioactivities exhibited by marine sulfated polysaccharides renders them ideal biomaterials for the development of novel systems for tissue engineering, wound healing, and drug delivery applications [M. Claveri, et al. Marine-derived polymeric materials and biomimetics: An overview. Polymers 2020, 12, 1002]. Carrageenans, ulvans and fucans possess anticoagulant, antioxidant, antitumor, antihyperlipidemic, as well as immunomodulating activities, while promoting attachment and proliferation of osteoblasts, presenting osteogenetic action K. Iliou, et al, Marine Biopolymers as Bioactive Functional Ingredients of Electrospun Nanofibrous Scaffolds for Biomedical Applications. Mar. Drugs 2022, 20, 314]. Possessing multifunctional characteristics and tunable mechanical properties, the electrospun nanofibrous scaffolds based on marine sulfated polysaccharides are attracting a constantly increasing interest for the development of advanced systems in the biomedical field [K. Iliou, et al, Marine Biopolymers as Bioactive Functional Ingredients of Electrospun Nanofibrous Scaffolds for Biomedical Applications. Mar. Drugs 2022, 20, 314].

In the prior art, there is no mention regarding compositions of dental micro/nanofibrous membranes, which simultaneously comprise marine sulfated polysaccharides and calcium, have the ability to promote bone and periodontal ligament regeneration and can comprise antibacterial agents.

The present invention refers to compositions in the form of a two- or three-layer membrane for periodontal use, which comprise an outer layer of hydrophobic polymers and one or two inner layers in the form of electrospun micro/nanofibers based on marine sulfated polysaccharides and calcium.

The above-mentioned problems are solved by the current invention defined by the following definitions:

Definition 1. Composition in the form of a two- or three-layer membrane for periodontal use, which comprises A, as well as B, wherein
A are hydrophobic polymers and
B are selected from the following four alternatives b₁ and/or b₂ and/or b₃ and/or b₄, wherein
b₁ is in the form of electrospun micro/nanofibers calcium salts of marine sulfated polysaccharides,
b₂ is in the form of electrospun micro/nanofibers a mixture of calcium salts of marine sulfated polysaccharides and calcium salts of hydrophilic polymers,
b₃ is in the form of electrospun micro/nanofibers a mixture of marine sulfated polysaccharides and calcium salts of marine sulfated polysaccharides,
b₄ is in the form of electrospun micro/nanofibers a mixture of marine sulfated polysaccharides and calcium salts of hydrophilic polymers.

Definition 2. Composition according to the definition 1, wherein the marine sulfated polysaccharides and/or calcium salts thereof included in b₁ or b₂ or b₃ or b₄ for the production of the electrospun micro/nanofibers are selected from carrageenans, ulvans and fucans and/or calcium salts thereof, preferably from carrageenans and/or calcium salts of carrageenans, more preferably are calcium salts of carrageenans.

Definition 3. Composition according to any of the definitions 1 to 2, wherein the calcium salts of hydrophilic polymers included in b₂ or b₄ are selected from calcium salts of poly(L-glutamic acid) and calcium salts of poly(L-aspartic acid), preferably are calcium salts of poly(L-glutamic acid).

Definition 4. Composition according to any of the definitions 1 to 3, wherein A is selected from polycaprolactone, poly(L-lactide) and poly(DL-lactide), preferably is polycaprolactone or poly(DL-lactide), more preferably is polycaprolactone.

Definition 5. Composition according to any of the definitions 1 to 4, which is formulated in two layers wherein i) the outer layer comprises A and ii) the inner layer comprises b₁ and/or b₂ and/or b₃ and/or b₄.

Definition 6. Composition according to the definition 5, wherein the average diameter of the electrospun micro/nanofibers of the inner layer ranges from 100 nm to 1100 nm, preferably from 300 nm to 900 nm, more preferably from 500 nm to 700 nm, for example 632 nm.

Definition 7. Composition according to any of the definitions 5 to 6, which is formulated into two layers wherein i) the outer layer comprises polycaprolactone, for example in the form of a cast film and ii) the inner layer comprises in the form of electrospun micro/nanofibers calcium salts of poly(L-glutamic acid) and calcium salts of carrageenans.

Definition 8. Composition according to any of the definitions 1 to 4, which is formulated in three layers wherein i) the outer layer comprises A, ii) the intermediate layer comprises b₁ and/or b₂ and/or b₃ and/or b₄ or calcium salts of hydrophilic polymers and iii) the inner layer comprises b₁ and/or b₂ and/or b₃ and/or b₄.

Definition 9. Composition according to the definition 8, wherein the average diameter of the electrospun micro/nanofibers of the intermediate and/or of the inner layer ranges from 40 nm to 600 nm, preferably from 100 nm to 500 nm, more preferably from 200 nm to 400 nm, for example 292 nm.

Definition 10. Composition according to any of the definitions 8 to 9, which is formulated into three layers, wherein i) the outer layer comprises polycaprolactone, for example in the form of a cast film, ii) the intermediate layer comprises in the form of electrospun micro/nanofibers calcium salts of poly(L-glutamic acid) and iii) the inner layer comprises in the form of electrospun micro/nanofibers calcium salts of carrageenans.

Definition 11. Composition according to any of the definitions 1 to 10, which additionally comprises one or more pharmaceutically active substances, preferably antibacterial agents, more preferably metronidazole or ciprofloxacin and most preferably metronidazole and/or other non-pharmaceutically active substances and/or other excipients.

Definition 12. Composition according to any of the definitions 1 to 11, for use in the treatment of periodontitis.

Prior to the present invention, there is no mention in the prior art regarding the use of the unique combination comprising an outer layer of hydrophobic polymers and one or two inner layers in the form of electrospun micro/nanofibers based on marine sulfated polysaccharides and calcium, in the specific forms of the above definitions.

It was surprisingly found that the composition of the present invention, as defined by definitions 1 to 12, comprising an outer layer of hydrophobic polymers and one or two inner layers in the form of electrospun micro/nanofibers based on marine sulfated polysaccharides and calcium, exhibits osteogenic activity due to the use of the marine sulfated polysaccharides and accelerates osteogenesis due to calcium.

In addition, it was surprisingly found that the composition of the present invention, as defined by definitions 1 to 12, comprising an outer layer of hydrophobic polymers and one or two inner layers in the form of electrospun micro/nanofibers based on marine sulfated polysaccharides and calcium, promotes cell adhesion of the periodontal ligament cells, osteogenic differentiation of the periodontal ligament cells and osteoblast proliferation, which can enhance bone and periodontal ligament regeneration.

It was surprisingly found that the composition of the present invention, as defined by definitions 1 to 12, comprising an outer layer of hydrophobic polymers and one or two inner layers in the form of electrospun micro/nanofibers based on marine sulfated polysaccharides and calcium, exhibits enhanced mechanical strength and acts as a barrier that prevents the proliferation of epithelial cells, due to the presence in the outer layer of hydrophobic polymers, for example in the form of a non-porous polymeric cast film.

Finally, it was surprisingly found that the more specific the scope of the claims is, the more intense the effects and the advantages of the present invention are.

According to the present invention, the composition in the form of a two- or three-layer membrane for periodontal use comprises an outer layer of hydrophobic polymers and one or two inner layers in the form of electrospun micro/nanofibers based on marine sulfated polysaccharides and calcium.

According to the present invention, in the composition in the form of a two- or three-layer membrane for periodontal use, the calcium salts of hydrophilic polymers for the production of electrospun micro/nanofibers are selected from the calcium salts of poly(L-glutamic acid) and the calcium salts of poly(L-aspartic acid).

In a preferred embodiment, in the composition in the form of two- or three-layer membrane for periodontal use of the present invention, the calcium salts of hydrophilic polymers for the production of electrospun micro/nanofibers are the calcium salts of poly(L-glutamic acid).

In a preferred embodiment, in the composition in the form of two- or three-layer membrane for periodontal use of the present invention, the calcium salts of hydrophilic polymers for the production of electrospun micro/nanofibers are the calcium salts of poly(L-aspartic acid).

According to the present invention, in the composition in the form of a two- or three-layer membrane for periodontal use, the marine sulfated polysaccharides or their calcium salts thereof for the production of electrospun micro/nanofibers are selected from carrageenans, ulvans and fucans and/or salts thereof.

In a preferred embodiment, in the composition in the form of a two- or three-layer membrane for periodontal use of the present invention, the marine sulfated polysaccharides for the production of electrospun micro/nanofibers are carrageenans.

In a preferred embodiment, in the composition in the form of a two- or three-layer membrane for periodontal use of the present invention, the salts of marine sulfated polysaccharides for the production of electrospun micro/nanofibers are the calcium salts of carrageenans.

In a preferred embodiment, in the composition in the form of a two- or three-layer membrane for periodontal use of the present invention, the marine sulfated polysaccharides for the production of electrospun micro/nanofibers are ulvans.

In a preferred embodiment, in the composition in the form of a two- or three-layer membrane for periodontal use of the present invention, the salts of marine sulfated polysaccharides for the production of electrospun micro/nanofibers are the calcium salts of ulvans.

In a preferred embodiment, in the composition in the form of a two- or three-layer membrane for periodontal use of the present invention, the marine sulfated polysaccharides for the production of electrospun micro/nanofibers are fucans.

In a preferred embodiment, in the composition in the form of a two- or three-layer membrane for periodontal use of the present invention, the salts of marine sulfated polysaccharides for the production of electrospun micro/nanofibers are the calcium salts of fucans.

According to the present invention, in the composition in the form of a two- or three-layer membrane for periodontal use of the present invention, the hydrophobic polymers form the outer layer of the composition, for example in the form of a cast film, and are selected from polycaprolactone, poly(L-lactide) and poly(DL-lactide).

In a preferred embodiment, the hydrophobic polymer in the composition in the form of a two- or three-layer membrane for periodontal use of the present invention is polycaprolactone.

In a preferred embodiment, the hydrophobic polymer in the composition in the form of a two- or three-layer membrane for periodontal use of the present invention is poly(L-lactide).

In a preferred embodiment, the hydrophobic polymer in the composition in the form of a two- or three-layer membrane for periodontal use of the present invention is poly(DL-lactide).

According to the present invention, the composition in the form of membrane for periodontal use of the present invention is formulated in two layers, wherein the outer layer comprises hydrophobic polymers and the inner layer comprises in the form of electrospun micro/nanofibers marine sulfated polysaccharides and calcium.

In a preferred embodiment, the composition in the form of membrane for periodontal use of the present invention is formulated in two layers, wherein the outer layer comprises hydrophobic polymers and the inner layer comprises in the form of electrospun micro/nanofibers calcium salts of marine sulfated polysaccharides.

In a preferred embodiment, the composition in the form of membrane for periodontal use of the present invention is formulated in two layers, wherein the outer layer comprises hydrophobic polymers and the inner layer comprises in the form of electrospun micro/nanofibers calcium salts of marine sulfated polysaccharides and calcium salts of hydrophilic polymers.

In a preferred embodiment, the composition in the form of membrane for periodontal use of the present invention is formulated in two layers, wherein the outer layer comprises hydrophobic polymers and the inner layer comprises in the form of electrospun micro/nanofibers marine sulfated polysaccharides and calcium salts thereof.

In a preferred embodiment, the composition in the form of membrane for periodontal use of the present invention is formulated in two layers, wherein the outer layer comprises hydrophobic polymers and the inner layer comprises in the form of electrospun micro/nanofibers marine sulfated polysaccharides and calcium salts of hydrophilic polymers.

In a particularly preferred embodiment, the composition in the form of membrane for periodontal use of the present invention is formulated in two layers, wherein the outer layer comprises polycaprolactone and the inner layer comprises in the form of electrospun micro/nanofibers calcium salts of carrageenan.

In a particularly preferred embodiment, the composition in the form of membrane for periodontal use of the present invention is formulated in two layers, wherein the outer layer comprises polycaprolactone and the inner layer comprises in the form of electrospun micro/nanofibers calcium salts of carrageenans and calcium salts of poly(L-glutamic acid).

In a particularly preferred embodiment, the composition in the form of membrane for periodontal use of the present invention is formulated in two layers, wherein the outer layer comprises polycaprolactone and the inner layer comprises in the form of electrospun micro/nanofibers carrageenans and calcium salts thereof.

In a particularly preferred embodiment, the composition in the form of membrane for periodontal use of the present invention is formulated in two layers, wherein the outer layer comprises polycaprolactone and the inner layer comprises in the form of electrospun micro/nanofibers carrageenans and calcium salts of poly(L-glutamic acid).

According to the present invention, in the composition in the form of membrane for periodontal use of the present invention that is formulated in two layers, the average diameter of the electrospun micro/nanofibers of the inner layer ranges from 100 nm to 1100 nm.

In a preferred embodiment, in the composition in the form of membrane for periodontal use of the present invention that is formulated in two layers, the average diameter of the electrospun micro/nanofibers of the inner layer ranges from 300 nm to 900 nm.

In a particularly preferred embodiment, in the composition in the form of membrane for periodontal use of the present invention that is formulated in two layers, the average diameter of the electrospun micro/nanofibers of the inner layer ranges from 500 nm to 700 nm, for example 632 nm.

According to the present invention, the composition in the form of a membrane for periodontal use of the present invention is formulated into three layers, wherein the outer layer comprises hydrophobic polymers, the intermediate layer and the inner layer comprise in the form of electrospun micro/nanofibers marine sulfated polysaccharides and calcium.

In a preferred embodiment, the composition in the form of a membrane for periodontal use of the present invention is formulated into three layers, wherein the outer layer comprises hydrophobic polymers, the intermediate layer comprises in the form of electrospun micro/nanofibers calcium salts of marine sulfated polysaccharides and the inner layer comprises in form of electrospun micro/nanofibers marine sulfated polysaccharides.

In a preferred embodiment, the composition in the form of a membrane for periodontal use of the present invention is formulated into three layers, wherein the outer layer comprises hydrophobic polymers, the intermediate layer comprises in the form of electrospun micro/nanofibers calcium salts of hydrophilic polymers and the inner layer comprises in form of electrospun micro/nanofibers marine sulfated polysaccharides.

In a preferred embodiment, the composition in the form of a membrane for periodontal use of the present invention is formulated into three layers, wherein the outer layer comprises hydrophobic polymers, the intermediate layer comprises in the form of electrospun micro/nanofibers calcium salts of marine sulfated polysaccharides and calcium salts of hydrophilic polymers and the inner layer comprises in form of electrospun micro/nanofibers marine sulfated polysaccharides.

In a preferred embodiment, the composition in the form of a membrane for periodontal use of the present invention is formulated into three layers, wherein the outer layer comprises hydrophobic polymers, the intermediate layer comprises in the form of electrospun micro/nanofibers calcium salts of marine sulfated polysaccharides and the inner layer comprises in form of electrospun micro/nanofibers calcium salts of marine sulfated polysaccharides.

In a preferred embodiment, the composition in the form of a membrane for periodontal use of the present invention is formulated into three layers, wherein the outer layer comprises hydrophobic polymers, the intermediate layer comprises in the form of electrospun micro/nanofibers calcium salts of hydrophilic polymers and the inner layer comprises in form of electrospun micro/nanofibers calcium salts of marine sulfated polysaccharides.

In a preferred embodiment, the composition in the form of a membrane for periodontal use of the present invention is formulated into three layers, wherein the outer layer comprises hydrophobic polymers, the intermediate layer comprises in the form of electrospun micro/nanofibers calcium salts of marine sulfated polysaccharides and calcium salts of hydrophilic polymers and the inner layer comprises in form of electrospun micro/nanofibers calcium salts of marine sulfated polysaccharides.

In a particularly preferred embodiment, the composition in the form of a membrane for periodontal use of the present invention is formulated into three layers, wherein the outer layer comprises polycaprolactone, the intermediate layer comprises in the form of electrospun micro/nanofibers calcium salts of carrageenans and the inner layer comprises in form of electrospun micro/nanofibers calcium salts of carrageenans.

In a particularly preferred embodiment, the composition in the form of a membrane for periodontal use of the present invention is formulated into three layers, wherein the outer layer comprises polycaprolactone, the intermediate layer comprises in the form of electrospun micro/nanofibers calcium salts of poly(L-glutamic acid) and the inner layer comprises in form of electrospun micro/nanofibers calcium salts of carrageenans.

In a particularly preferred embodiment, the composition in the form of a membrane for periodontal use of the present invention is formulated into three layers, wherein the outer layer comprises polycaprolactone, the intermediate layer comprises in the form of electrospun micro/nanofibers calcium salts of poly(L-glutamic acid) and calcium salts of carrageenans and the inner layer comprises in form of electrospun micro/nanofibers calcium salts of carrageenans.

In a particularly preferred embodiment, the composition in the form of a membrane for periodontal use of the present invention is formulated into three layers, wherein the outer layer comprises polycaprolactone, the intermediate layer comprises in the form of electrospun micro/nanofibers calcium salts of carrageenans and the inner layer comprises in form of electrospun micro/nanofibers carrageenans.

In a particularly preferred embodiment, the composition in the form of a membrane for periodontal use of the present invention is formulated into three layers, wherein the outer layer comprises polycaprolactone, the intermediate layer comprises in the form of electrospun micro/nanofibers calcium salts of poly(L-glutamic acid) and the inner layer comprises in form of electrospun micro/nanofibers carrageenans.

In a particularly preferred embodiment, the composition in the form of a membrane for periodontal use of the present invention is formulated into three layers, wherein the outer layer comprises polycaprolactone, the intermediate layer comprises in the form of electrospun micro/nanofibers calcium salts of poly(L-glutamic acid) and calcium salts of carrageenans and the inner layer comprises in form of electrospun micro/nanofibers carrageenans.

According to the present invention, in the composition in the form of membrane for periodontal use of the present invention that is formulated into three layers, the average diameter of the electrospun micro/nanofibers of the intermediate and/or of the inner layer ranges from 40 nm to 600 nm.

In a preferred embodiment, in the composition in the form of membrane for periodontal use of the present invention that is formulated into three layers, the average diameter of the electrospun micro/nanofibers of the intermediate and/or of the inner layer ranges from 100 nm to 500 nm.

In a particularly preferred embodiment, in the composition in the form of membrane for periodontal use of the present invention that is formulated into three layers, the average diameter of the electrospun micro/nanofibers of the intermediate and/or of the inner layer ranges from 200 nm to 400 nm, for example 292 nm.

According to the present invention, the composition in the form of a two- or three-layer membrane for periodontal use of the present invention comprises one or more pharmaceutically active substances and/or other non-pharmaceutically active substances and/or other excipients.

In a preferred embodiment, the composition in the form of a two- or three-layer membrane for periodontal use of the present invention comprises antibacterial agents.

In a particularly preferred embodiment, the composition in the form of a two- or three-layer membrane for periodontal use of the present invention comprises metronidazole.

In a particularly preferred embodiment, the composition in the form of a two- or three-layer membrane for periodontal use of the present invention comprises ciprofloxacin.

According to the present invention, the composition in the form of a two- or three-layer membrane for periodontal use of the present invention is suitable for use in the treatment of periodontitis or other diseases in the oral cavity.

The present invention is further described by the following indicative, non-limiting examples:

### EXAMPLE 1: PREPARATION OF MICRO/NANOFIBROUS MEMBRANE FOR PERIODONTAL USE IN THE FORM OF THREE LAYERS (GTR1)

The outer layer comprises a cast polycaprolactone film, the intermediate layer comprises in the form of electrospun micro/nanofibers calcium salts of poly(L-glutamic acid), while the inner layer comprises in the form of electrospun micro/nanofibers calcium salts of carrageenan. For the electrospinning of the calcium salts of poly(L-glutamic acid) (PG-Ca) and the calcium salts of carrageenan (CG-Ca), polyethylene oxide (PEO) was used as a hydrophilic polymeric carrier, in order to enhance their electrospinnability in non-woven membranes.

### 1. Preparation of Spinning Solutions

All spinning solutions were prepared under stirring for 24 h to ensure their homogeneity. The spinning solution of polycaprolactone (PCL) was prepared at room temperature, whereas all the aqueous spinning solutions were prepared at 60° C. For the preparation of the PCL spinning solution, PCL (molecular weight 80,000) was dissolved at a concentration of 10% w/v in DCM/DMF 8:2 (for example, 0.27 g of PCL in 2.7 mL of the solvent). The CG-Ca/PEO spinning solution was prepared by dissolving CG-Ca at a concentration of 1% w/v in distilled H₂O followed by the addition of PEO (molecular weight 900,000) at a concentration of 4% w/v (for example, 0.05 g of CG-Ca and 0.2 g PEO in 5 mL of the solvent). The PG-Ca/PEO spinning solution was prepared by dissolving PG-Ca at a concentration of 4% w/v in distilled H₂O followed by the addition of PEO (molecular weight 7,000,000) at a concentration of 4% w/v (for example, 0.2 g of PG-Ca and 0.2 g PEO in 5 mL of the solvent).

Electrospinning was conducted using a y-High Voltage Research DC power supply generator of 50 kV maximum voltage (Gamma High Voltage Research) with the spinning solutions being loaded into 10 mL disposable syringes fitted with stainless steel blunt needles of 23 gauge diameter (23G). The syringes were mounted on a horizontally positioned programmable syringe pump (Harvard PHD 2000, Harvard Apparatus) and the produced micro/nanofibers were deposited on a RC-6000 (NaBondTechnologies) rotating drum collector at a rotation speed of 400 rpm. Temperature and relative humidity were 21±2° C. and 60±5%, respectively.

### 2. Preparation of the Outer Layer

For the preparation of the outer layer the process of solvent casting was followed so as to a create a cast membrane on the surface of a rotating drum. To prepare the outer layer (cast membrane), PCL (molecular weight 80,000) was dissolved at a concentration of 8% w/v in benzene (for example, 24 g of PCL in 300 mL of the solvent) at room temperature under stirring for 48 h to ensure the homogeneity of the solution. An appropriate volume of the PCL solution was transferred to a container. Subsequently, a drum was dipped in the polymer solution and allowed to rotate until the entire surface of the cylinder was covered by the polymer solution. Then the vessel containing the PCL solution was removed and the drum was allowed to rotate over the air until complete evaporation of the solvent and the formation of a thin membrane on its surface. The outer layer served as the control sample (CTRL) in the various analyses of the fabricated multi-layer membranes.

### 3. Fabrication of the Middle Layer

For the fabrication of the middle layer of the tri-layer membrane GTR1, the middle layer was electrospun on the surface of the outer layer (PCL cast membrane). To obtain the middle layer, the PCL and PG-Ca/PEO spinning solutions were co-electrospun using an antiparallel setup with the syringes mounted on two horizontally opposed programmable syringe pumps to ensure the homogeneity of the blended polymer fibers (to ensure the cohesion between the deposited fibers and the cast film, a small volume of the PCL electrospinning solution was first electrospun separately and then the electrospinning solutions were co-electrospun). The feeding rate and tip-to-collector distance of the PCL spinning solution was fixed at 2.5 mL/h and 16 cm, respectively, whereas the feeding rate and tip-to-collector distance of the PG-Ca/PEO spinning solution was adjusted at 5 mL/h and 27 cm, respectively. Electrospinning was conducted with the applied voltage fixed at 27 kV.

### 4. Fabrication of the Inner Layer

For the fabrication of the inner layer of the tri-layer membrane GTR1, the inner layer was electrospun on the surface of the middle layer. To obtain the inner layer, the CG-Ca/PEO spinning solution was electrospun with the applied voltage, the feeding rate and tip-to-collector distance fixed at 27 kV, 3 mL/h and 30 cm, respectively.

### EXAMPLE 2: PREPARATION OF MICRO/NANOFIBROUS MEMBRANE FOR PERIODONTAL USE IN THE FORM OF TWO LAYERS (GTR2)

The outer layer comprises a cast film of polycaprolactone, while the inner layer comprises in the form of electrospun micro/nanofibers calcium salts of poly(L-glutamic acid) and calcium salts of carrageenan. For the electrospinning of the calcium salts of poly(L-glutamic acid) (PG-Ca) and the calcium salts of carrageenan (CG-Ca), polyethylene oxide (PEO) was used as a hydrophilic polymeric carrier in order to enhance their electrospinnability in non-woven membranes.

### 1. Preparation of Spinning Solutions

All spinning solutions were prepared under stirring for 24 h to ensure their homogeneity. The spinning solution of PCL was prepared at room temperature, whereas all the aqueous spinning solutions CG-Ca/PG-Ca/PEO were prepared at 60° C. For the preparation of the PCL spinning solution, PCL (molecular weight 80,000) was dissolved at a concentration of 10% w/v in DCM/DMF 8:2 (for example, 0.27 g of PCL in 2.7 mL of the solvent). The CG-Ca/PG-Ca/PEO spinning solution was prepared by dissolving CG-Ca at a concentration of 1% w/v in distilled H2O followed by the addition of PG-Ca at a concentration of 4% w/v and PEO (molecular weight 7,000,000) at a concentration of 4% w/v (for example, 0.05 g of CG-Ca, 0.2 g of PG-Ca and 0.2 g PEO in 5 mL of the solvent).

Electrospinning was conducted using a y-High Voltage Research DC power supply generator of 50 kV maximum voltage (Gamma High Voltage Research) with the spinning solutions being loaded into 10 mL disposable syringes fitted with stainless steel blunt needles of 23 gauge diameter (23G). The syringes were mounted on a horizontally positioned programmable syringe pump (Harvard PHD 2000, Harvard Apparatus) and the produced micro/nanofibers were deposited on a RC-6000 (NaBondTechnologies) rotating drum collector at a rotation speed of 400 rpm. Temperature and relative humidity were 21±2° C. and 60±5%, respectively.

### 2. Preparation of the Outer Layer

The outer layer of the membrane in the form of two layers is prepared as previously described for the membrane in the form of three layers in Example 1.

### 3. Fabrication of the Inner Layer

For the fabrication of the inner layer of the bi-layer membrane GTR2, the inner layer was electrospun on the surface of the outer layer (PCL cast membrane). In order to get the inner layer, the PCL and CG-Ca/PG-Ca/PEO spinning solutions were co-electrospun using an antiparallel setup with the syringes mounted on two horizontally opposed programmable syringe pumps to ensure the homogeneity of the blended polymer fibers (to ensure the cohesion between the deposited fibers and the cast film, a small volume of the PCL electrospinning solution was first electrospun separately and then the electrospinning solutions were co-electrospun). Electrospinning was conducted with the applied voltage fixed at 27 kV. The feeding rate and tip-to-collector distance of the PCL spinning solution was fixed at 2.5 mL/h and 16 cm, respectively, whereas the feeding rate and tip-to-collector distance of the CG-Ca/PG-Ca/PEO spinning solution was adjusted at 5 mL/h and 27 cm, respectively.

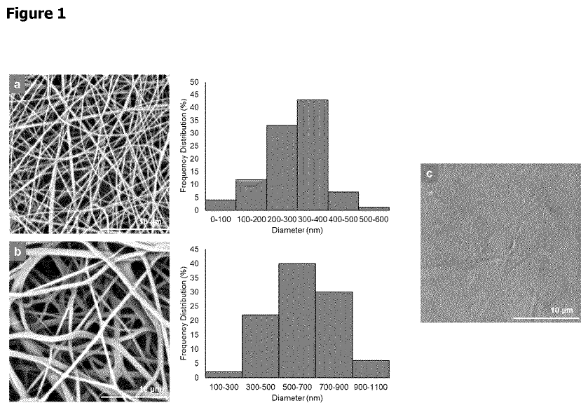

The micro/nanofibrous membranes GTR1 and GTR2 of Examples 1 and 2 were characterized for their morphology, diameter range, and average diameter of the micro/nanofibers. The morphological characterization of the micro/nanofibrous membranes GTR1 and GTR2 of Examples 1 and 2 (FIG. 1) was performed using a PhenomWorld (Thermo Fischer Scientific) desktop scanning electron microscope (SEM) with tungsten filament (10 kV) and a charge reduction sample holder. It was shown that a non-porous surface was obtained in the outer layer, whereas a uniform micro/nanofibrous network was successfully obtained with fibers of cylindrical morphology in the inner electrospun layers of both fabricated membranes. The range of diameters and the average diameters of the produced micro/nanofibers are shown in Table 1.

TABLE 1
Range of diameter and average diameter
of the produced micro/nanofibers.

| Membranes | Range of fiber diameter | Average fiber diameter |
|---|---|---|
| GTR1 | 43 nm-586 nm | 292 ± 93 nm |
| GTR2 | 154 nm-1180 nm | 632 ± 176 nm |

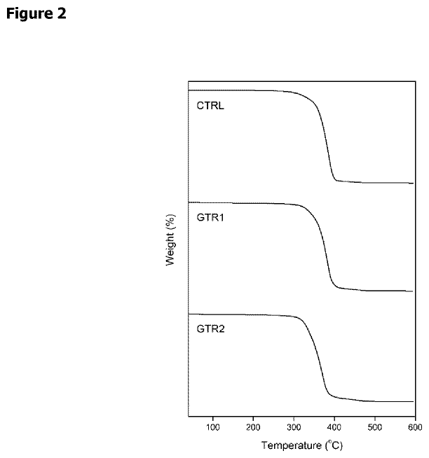

The micro/nanofibrous membranes GTR1 and GTR2 of Examples 1 and 2 were characterized for their thermal stability. The thermal stability of the micro/nanofibrous membranes GTR1 and GTR2 of Examples 1 and 2 was investigated by thermogravimetric analysis (TGA) using a TA Thermogravimetric Analyzer (TGA 55, TA Instruments). As shown in the TGA thermograms (FIG. 2), the CTRL, GTR1 and GTR2 membranes displayed similar thermal profiles characterized by a main degradation step in their thermogravimetric curves. Specifically, the degradation of CTRL was initiated at 335 and completed at 401° C. The decomposition of GTR1 was initiated at 319° C. and completed at 403° C., whereas GTR2 started to decompose at 309° C. and its degradation was completed at around 391° C.

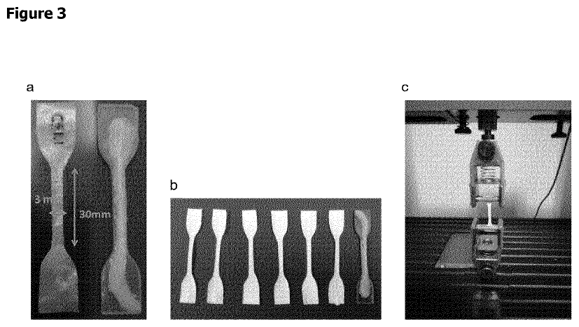

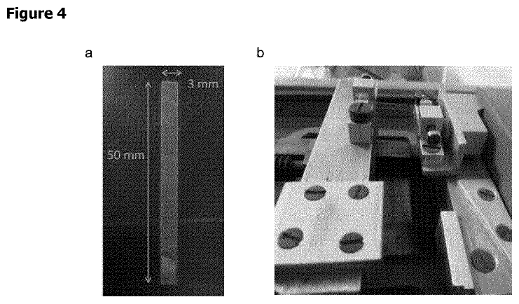

The major mechanical properties of the micro/nanofibrous membranes GTR1 and GTR2 of Examples 1 and 2, as well as the membrane reference CTRL, were tested utilizing specifically prepared specimens. The mechanical properties determined included the modulus of elasticity (E), the ultimate tensile strength (UTS), the plastic deformation and the percentile reduction of initial force (RAS). Seven dumbbell shaped specimens of specific dimensions were cut off for each membrane and placed in a tensometer (Tensometer Aegis 10, Monsanto, UK) (FIG. 3). The cross head speed was set at 25 mm/min. The results were recorded and stress-strain diagrams were produced from which the membrane resistance to elastic deformation, as well as the tensile strength and plastic deformation were calculated. As far as the determination of force reduction under constant strain (relaxation) is concerned, seven orthogonal parallelogram shaped specimens were prepared for each membrane and then placed in a low force load cell machine connected to a PC measuring force every 30 seconds (FIG. 4). The initially applied force was set at 3N and its reduction in time was recorded for 48 h since initial studies indicated that the plateau phase was reached much sooner. The collected data from both tests were statistically assessed utilizing one-way ANOVA and Tukey-test.

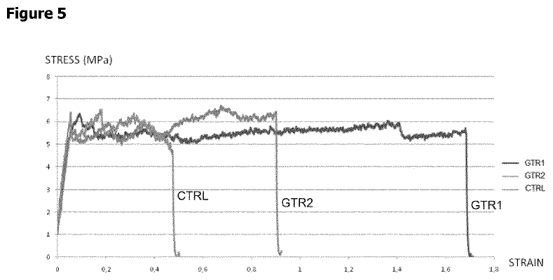

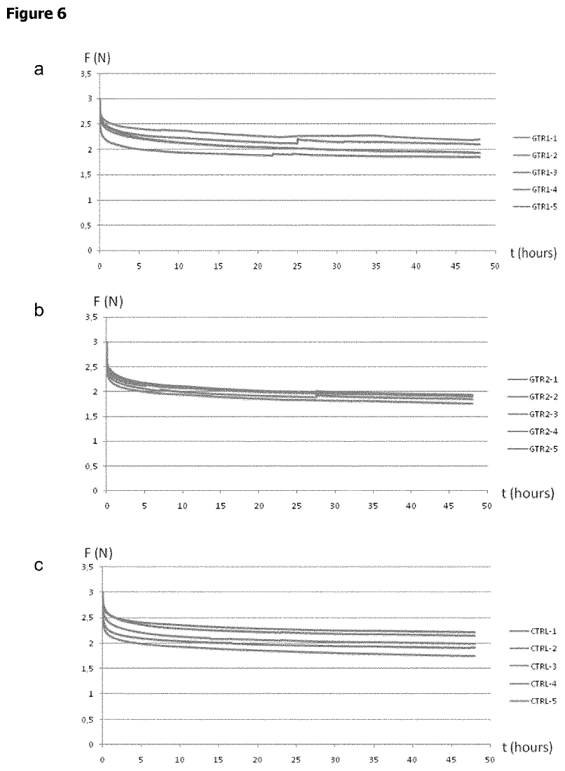

The results on the mechanical properties of the micro/nanofibrous membranes GTR1 and GTR2 of Examples 1 and 2 are presented in Table 2 and depicted in the corresponding diagrams of FIG. 5 and FIG. 6. The two-layer membrane GTR2 is characterized by the highest value of modulus of elasticity (128.3 MPa) followed by the CTRL membrane serving as control and lastly the three-layer membrane GTR1. Although, the CTRL membrane presents the lowest mean value of ultimate tensile strength, this is not significantly different from the other two. From the stress strain curves it is obvious that the placement of further layers upon the CTRL membrane results in a dramatic increase of plastic deformation ranging from 1.6 to 3.5 times. As far as relaxation testing is concerned, no statistically significant differences were recorded among the membranes. The percentile reduction of the initial force under constant strain in 48 h reached 35%, with 80% of it taking place the first hours.

In conclusion, the membrane GTR2 presented the highest mean value of modulus of elasticity. The differences of the mean values of modulus of elasticity and ultimate tensile strength among the different membranes are not decisively high. The control membrane CTRL is the major factor affecting the basic mechanical properties of E and UTS. The addition of further layers on the CTRL membrane increases plastic deformation. The membranes percentile reduction of initial force under constant strain is intensive during the first hours and no statistically significant difference is observed in 48 h between the means of the different membranes.

TABLE 2
Mechanical properties of the micro/nanofibrous membranes
GTR1 and GTR2 of Examples 1 and 2, as well as of membrane
CTRL, presented as mean values (standard deviation).

| | | | Plastic | |
|---|---|---|---|---|
| Periodontal | | UTS | deformation | |
| membranes | E (MPa) | (MPa) | (%) | RAS (%) |
| GTR1 | 88.0(20.9)^{A} | 6.8(2.5)^{A} | 161.2(74.6) | 33.4(4.8)^{A} |
| GTR2 | 128.3(26.8)^{B} | 6.5(1.1)^{A} | 74.4(14.8) | 37.7(2.3)^{A} |
| CTRL | 91.0(20.1)^{A} | 5.7(1.1)^{A} | 46.3(8.7) | 33.3(6.3)^{A} |

E (modulus of elasticity), UTS (ultimate tensile strength) and RAS (percentile reduction of initial force under constant strain). The presence of different abbreviation letters indicate statistically significant differences between the means of the different membranes (p<0.05).

The micro/nanofibrous membranes GTR1 and GTR2 of Examples 1 and 2 were evaluated regarding their cell attachment and the morphology of PDL cells seeded on the fabricated membranes. The micro/nanofibrous membranes GTR1 and GTR2 of Examples 1 and 2 were evaluated in vitro using human PDL cells that were isolated from the periodontal ligament of healthy-teeth and cultured in Dulbecco's Modified Eagle's Medium (DMEM). At passage 4, PDL cells were seeded onto the membranes GTR1, GTR2 and CTRL, or on plain culture plates (control) and cultured in the same medium.

Cells' attachment on the membranes was examined by scanning electron microscopy. The biocompatibility of these membranes for PDL cells was examined by MTT assay through days 1 to 7. The osteo-inductive properties of the membranes were studied by the Alizarin red staining.

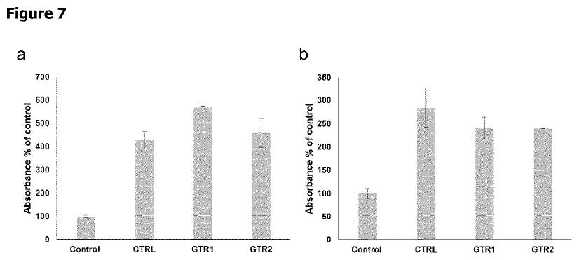

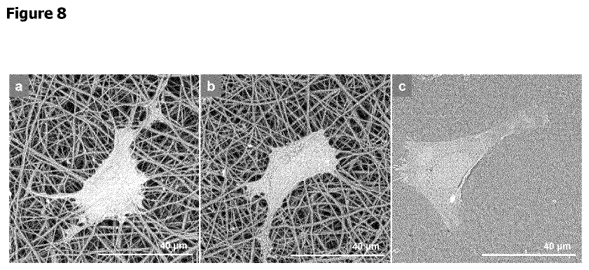

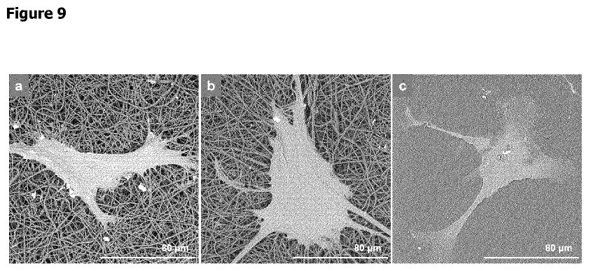

All examined membranes were biocompatible with PDL cells and non-cytotoxic (FIG. 7). The membranes were found to promote the attachment of the seeded cells (FIGS. 8 and 9).

The cells seeded on the membranes exhibited lamellipodia and filopodia extensions, a fact which is indicative of good cellular attachment and migration.

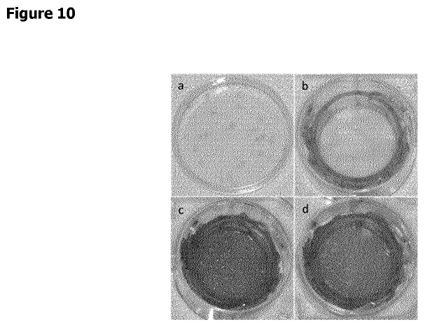

The Alizarin red staining showed that the membranes GTR1 and GTR2 of the Examples 1 and 2 (the former more than the latter) are able to promote osteogenic differentiation of PDL cells earlier (at 1 week in osteoinductive medium), as compared to the membrane CTRL and the control plate (FIG. 10).

From the above examples it can be seen that the said compositions present the advantages: increased mechanical strength and in particular a dramatic increase in plastic deformation compared to the reference membrane, they are biocompatible with periodontal ligament cells and non-cytotoxic, they have the ability to promote the adhesion and the migration of the periodontal ligament cells, as well as to promote the osteogenic differentiation of these cells. The supply of calcium from the compositions of Examples 1 and 2 has a significant positive contribution to the regeneration and osteodifferentiation of the periodontal ligament cells.

With reference to the following attached FIGS. 1 to 10 the above results are described in more detail and the above-indicated results are better understood:

FIG. 1. (a) SEM image and diameter distribution histogram of the membrane GTR1 with average diameter 292 nm, (b) SEM image and diameter distribution histogram of the membrane GTR2 with average diameter 632 nm and (c) SEM image of the membrane CTRL.

FIG. 2. TGA thermograms of the CTRL, GTR1 and GTR2 membranes.

FIG. 3. (a,b) Dumbbell shaped membrane specimens prepared utilizing an acrylic mould of specific dimensions for the tensile testing and (c) the illustration of the membranes being placed at the tensometer grabbers that moved apart at a speed of 25 mm/min.

FIG. 4. (a) Relaxation membrane specimens of orthogonal parallelogram shape (3×50 mm) and (b) Low force load cell that strains the membranes until an initial force of 3N is reached and then records its reduction in time.

FIG. 5. Indicative stress strain curves of the micro/nanofibrous membranes GTR1 and GTR2 of Examples 1 and 2, as well as of the reference membrane CTRL.

FIG. 6. Curves of relaxation test of (a) the micro/nanofibrous membrane GTR1 of Example 1, (b) the micro/nanofibrous membrane GTR2 of Example 2 and (c) the reference membrane CTRL.

FIG. 7. Proliferation of PDL cells seeded on plain culture plate (control) and on the CTRL, GTR1 and GTR2 membranes that were cultured for (a) 1 day and (b) 7 days.

FIG. 8. SEM images of PDL cells seeded on (a) GTR1, (b) GTR2 and (c) CTRL membranes after 4 days culture in DMEM.

FIG. 9. SEM images of PDL cells seeded on (a) GTR1, (b) GTR2 and (c) CTRL membranes after 4 days culture in osteogenic medium.

FIG. 10. Images of Alizarin red staining (dark grey on image) of PDL cells after osteogenic induction for 7 days, wherein (a) control, (b) CTRL membrane, (c) GTR1 membrane and (d) GTR2 membrane.

1. A composition in the form of a two- or three-layer membrane for periodontal use, which comprises A, as well as B, wherein
A are hydrophobic polymers and
B are selected from the following four alternatives b₁ and/or b₂ and/or b₃ and/or b₄, wherein
b₁ is in the form of electrospun micro/nanofibers calcium salts of marine sulfated polysaccharides,
b₂ is in the form of electrospun micro/nanofibers a mixture of calcium salts of marine sulfated polysaccharides and calcium salts of hydrophilic polymers,
b₃ is in the form of electrospun micro/nanofibers a mixture of marine sulfated polysaccharides and calcium salts of marine sulfated polysaccharides,
b₄ is in the form of electrospun micro/nanofibers a mixture of marine sulfated polysaccharides and calcium salts of hydrophilic polymers.

2. The composition according to claim 1, wherein the marine sulfated polysaccharides and/or calcium salts thereof included in b₁ or b₂ or b₃ or b₄ for the production of the electrospun micro/nanofibers are selected from carrageenans, ulvans and fucans and/or calcium salts thereof.

3. The composition according to claim 1, wherein the calcium salts of hydrophilic polymers included in b₂ or b₄ are selected from calcium salts of poly(L-glutamic acid) and calcium salts of poly(L-glutamic acid).

4. The composition according to claim 1, wherein A is selected from polycaprolactone, poly(L-lactide) and poly(DL-lactide).

5. The composition according to claim 1, which is formulated in two layers wherein i) the outer layer comprises A and ii) the inner layer comprises b₁ and/or b₂ and/or b₃ and/or b₄.

6. The composition according to claim 5, wherein the average diameter of the electrospun micro/nanofibers of the inner layer ranges from 100 nm to 1100 nm.

7. The composition according to claim 5, which is formulated into two layers wherein i) the outer layer comprises polycaprolactone, and ii) the inner layer comprises in the form of electrospun micro/nanofibers calcium salts of poly(L-glutamic acid) and calcium salts of carrageenans.

8. The composition according to claim 1, which is formulated in three layers wherein i) the outer layer comprises A, ii) the intermediate layer comprises b₁ and/or b₂ and/or b₃ and/or b₄ or calcium salts of hydrophilic polymers and iii) the inner layer comprises b₁ and/or b₂ and/or b₃ and/or b₄.

9. The composition according to claim 8, wherein the average diameter of the electrospun micro/nanofibers of the intermediate and/or of the inner layer ranges from 40 nm to 600 nm.

10. The composition according to claim 8, which is formulated into three layers, wherein i) the outer layer comprises polycaprolactone, ii) the intermediate layer comprises in the form of electrospun micro/nanofibers calcium salts of poly(L-glutamic acid) and iii) the inner layer comprises in the form of electrospun micro/nanofibers calcium salts of carrageenans.

11. The composition according to claim 1, which additionally comprises one or more pharmaceutically active substances.

12. The composition according to claim 1, for use in the treatment of periodontitis.
